(19) United States
(12) Patent Application Publication
(10) Pub. No.: US 2025/0104842 A1
(43) Pub. Date: Mar. 27, 2025

(54) METHOD AND SYSTEM FOR SHARING DIGITAL PATHOLOGICAL IMAGE

(71) Applicant: Wistron Medical Technology Corporation, HSINCHU CITY (TW)

(72) Inventors: Yi Sheng Chao, Hsinchu City (TW); Shih Feng Huang, Hsinchu City (TW); Chun Yi Wu, Hsinchu City (TW); Chao Yi Huang, Hsinchu City (TW); Han Chun Kuo, Hsinchu City (TW)

(73) Assignee: Wistron Medical Technology Corporation, HSINCHU CITY (TW)

(21) Appl. No.: 18/490,766

(22) Filed: Oct. 20, 2023

(30) Foreign Application Priority Data
Sep. 22, 2023 (TW) 112136229

(51) Int. Cl. G16H 30/20; G06F 3/0482; G06F 3/04845; G06F 3/14; G16H 30/40

(52) U.S. Cl. CPC G16H 30/20; G06F 3/0482; G06F 3/04845; G06F 3/1454; G16H 30/40; G06F 2203/04803

(57) ABSTRACT

A method and a system for sharing a digital pathological image are provided, where the digital pathological image is presented on a first interactive interface of a first terminal device with an editing permission and a second interactive interface of a second terminal device with an observing permission by a server. In the first terminal device, in response to an image adjustment operation or a tag adding operation performed through the first interactive interface, corresponding image adjustment information or added tag information is transmitted to the second interactive interface of the second terminal device through the first interactive interface. In the second terminal device, in response to receiving the image adjustment information or the added tag information through the second interaction interface, the content presented on the second interaction interface is correspondingly adjusted based on the image adjustment information or the tag adding information.

### CROSS-REFERENCE TO RELATED APPLICATION

This application claims the priority benefit of Taiwan patent application no. 112136229, filed on Sep. 22, 2023. The entirety of the above-mentioned patent application is hereby incorporated by reference herein and made a part of this specification.

### BACKGROUND

### Technical Field

The disclosure relates to a remote conferencing mechanism; more particularly, the disclosure relates to a method and a system for sharing digital pathological images.

### Description of Related Art

In the realm of remote collaborative synchronization, conventional video software falls short in its ability to display medical images in the standardized Digital Imaging and Communications in Medicine (DICOM) format. Enabling seamless multi-party synchronous interaction necessitates the elimination of any latency during such collaborative processes. These are the challenges that arise when applying remote collaborative synchronization in a medical context. Besides, the capacity to acquire expert medical opinions from diverse specialties based on the visual characteristics of medical images presented on a web viewer and to facilitate collaborative discourse to arrive at a diagnosis is a capability conspicuously absent within current web browsers.

Moreover, data compatibility for annotating nidus locations on medical images is also a highlighted challenge at present. For instance, due to the multitude of image formats and compression techniques employed for the storage of pathological images, tagging software cannot provide the comprehensive support for diverse image formats. Additionally, If the results of the nidus annotation are to be presented on terminal devices used by different observers, data conversion is required to obtain the corresponding content.

### SUMMARY

The disclosure provides a method and a system for sharing digital pathological images, which may be used in the application of remote consultation and collaborative synchronization and may allow multi-party synchronous interactions and ensure smooth image transmissions.

According to an embodiment of the disclosure, a method for sharing a digital pathological image includes following steps. A remote interactive service is provided to a first terminal device and a second terminal device through a server, where the first terminal device includes a first interactive interface, the second terminal device includes a second interactive interface, the first terminal device has an editing permission, and the second terminal device has an observing permission. The digital pathological image is presented to the first interactive interface and the second interactive interface through the server. In response to receiving an image adjustment operation performed on the digital pathological image through the first interactive interface, image adjustment information corresponding to the image adjustment operation is transmitted to the second interactive interface of the second terminal device through the first interactive interface. In response to receiving a tag adding operation performed on the digital pathological image through the first interactive interface, added tag information corresponding to the tag adding operation is transmitted to the second interactive interface of the second terminal device through the first interactive interface. In the second terminal device, in response to receiving the image adjustment information through the second interactive interface, the digital pathological image presented on the second interactive interface is adjusted based on the image adjustment information. In the second terminal device, in response to receiving the added tag information through the second interactive interface, a tag corresponding to the tag adding operation performed through the first interactive interface is added to the digital pathological image presented on the second interactive interface based on the added tag information.

According to an embodiment of the disclosure, a system for sharing a digital pathological image includes a server that provides a remote interactive service, a first terminal device that has an editing permission and includes a first interactive interface provided by the remote interactive service, and a second terminal device that has an observing permission and includes a second interactive interface provided by the remote interactive service. The server presents the digital pathological image to the first interactive interface and the second interactive interface. The first terminal device is configured to: in response to receiving an image adjustment operation performed on the digital pathological image through the first interactive interface, transmit image adjustment information corresponding to the image adjustment operation to the second interactive interface of the second terminal device through the first interactive interface; in response to receiving a tag adding operation performed on the digital pathological image through the first interactive interface, transmit added tag information corresponding to the tag adding operation to the second interactive interface of the second terminal device through the first interactive interface. The second terminal device is configured to: in response to receiving the image adjustment information through the second interactive interface, adjust the digital pathological image presented on the second interactive interface based on the image adjustment information; in response to receiving the added tag information through the second interactive interface, add a tag corresponding to the tag adding operation performed through the first interactive interface to the digital pathological image presented on the second interactive interface based on the added tag information.

Based on the above, the method and the system for sharing the digital pathological images as provided in one or more embodiments of the disclosure may be used in the application of remote consultation and collaborative synchronization and may allow multi-party synchronous interactions and ensure smooth image transmissions.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a schematic view illustrating a system for sharing a digital pathological image according to an embodiment of the disclosure.

FIG. 2 is a flowchart illustrating a method for sharing a digital pathological image according to an embodiment of the disclosure.

FIG. 3 is a flowchart illustrating a method of operating collaborative synchronization according to a first embodiment of the disclosure.

FIG. 4 is a flowchart illustrating a method of operating collaborative synchronization according to a second embodiment of the disclosure.

FIG. 5 is a schematic view illustrating an interactive interface according to an embodiment of the disclosure.

FIG. 6 is a schematic view illustrating divided windows according to an embodiment of the disclosure.

FIG. 7 is a schematic view illustrating an interactive interface having an editing permission according to an embodiment of the disclosure.

FIG. 8 is a schematic view illustrating an interactive interface having no editing permission according to an embodiment of the disclosure.

FIG. 9 is a schematic view illustrating a poll menu according to an embodiment of the disclosure.

FIG. 10 is a schematic view illustrating a poll result according to an embodiment of the disclosure.

### DETAILED DESCRIPTION OF THE EMBODIMENTS

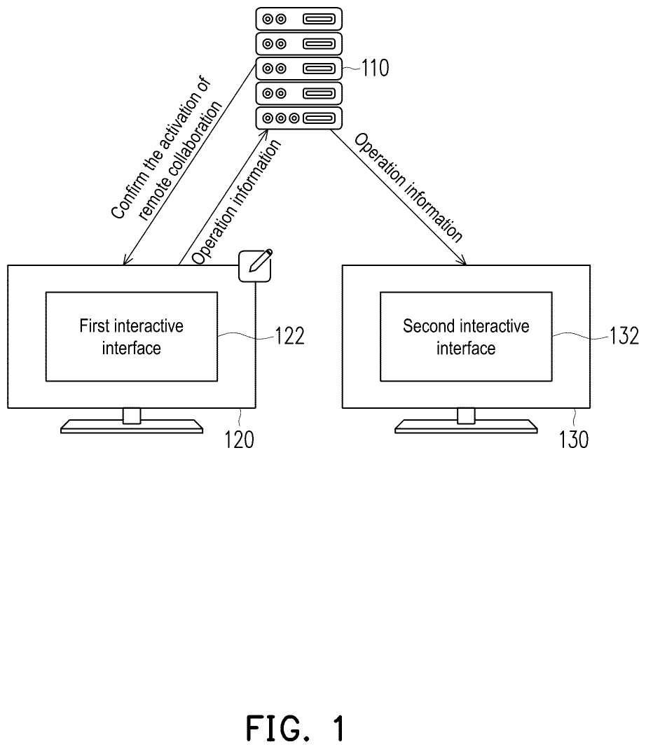

FIG. 1 is a schematic view illustrating a system for sharing a digital pathological image according to an embodiment of the disclosure. With reference to FIG. 1, the system simultaneously communicates with more than two terminal devices, e.g., a first terminal device 120 and a second terminal device 130, through a server 110. Here, the first terminal device 120 and the second terminal device 130 are explained, which should however not be construed as a limitation in the disclosure. In other embodiments, the number of the terminal devices may be three or more. The terminal devices may be smart mobile devices, tablet computers, desktop computers, or other electronic devices with computing and networking capabilities. The terminal devices include processors, communication components, storage devices, displays, and so on. The server 110 includes a processor, communication components, and a storage device.

The processor, for instance, may include a central processing unit (CPU), a physics processing unit (PPU), a programmable microprocessor, an embedded control chip, a digital signal processor (DSP), an application specific integrated Circuit (ASIC), or any other similar device.

The storage device, for instance, may include any type of fixed or removable random access memory (RAM), read-only memory (ROM), flash memory, hard disk, other similar devices, or a combination of these devices. The storage device stores one or more code snippets, which are executed by the processor after being installed.

The communication component may include a chip or a circuit that adopts a local area network (LAN) technology, a wireless LAN (WLAN) technology, or a mobile communication technology. The LAN, for instance, may be Ethernet. The WLAN, for instance, may be Wi-Fi. The mobile communication technology may, for instance, refer to global system for mobile communications (GSM), third-generation (3G) communications, fourth-generation (4G) communications, fifth-generation (5G) communications, and so forth.

The display, for instance, may include a liquid crystal display (LCD), a plasma display, and so on.

The server 110 is configured to provide a remote interactive service to the first terminal device 120 and the second terminal device 130 for use. The server 110, for instance, may include a computer with powerful computing capabilities and/or large storage space, which may complete a significant amount of work and store a considerable amount of data in a short period of time, and provide services to a number of users. In an embodiment, the remote interactive service may include, for instance, Socket.io. Socket.io mainly uses the WebSocket protocol. The server 110 switches between an editor and an observer among a plurality of terminal devices (at least including the first terminal device 120 and the second terminal device 130) through remote communications. At the same time, only one terminal device (the editor) has an editing permission, while the other terminal devices (the observers) do not have the editing permission but an observing permission and may simply observe operation results of the editor.

In this embodiment, it is assumed a meeting creator (editor) connects the server 110 through any electronic device (which may be the first terminal device 120, the second terminal device 130, or any other device) to set up a remote meeting and then lists a member list to select one or more participants. The server 110 may generate a meeting link and send the meeting link to communication addresses (such as email accounts, text messages, and so on) corresponding to the selected participants. The creator and the participants click on the meeting link through the terminal devices they use, thereby joining the same remote meeting through the server 110, and the server 110 provides an interactive interface to each terminal device connected to the server 110.

Here, the server 110 is assumed to have preset the first terminal device 120 to have an editing permission, while the second terminal device 130 does not have the editing permission. The server 110 provides the first interactive interface 122 to the first terminal device 120, and provides the second interactive interface 132 to the second terminal device 130.

In an embodiment of the disclosure, the first terminal device 120 and the second terminal device 130 may join the remote meeting created by the server 110 through their internally installed browsers. In another embodiment of the disclosure, an application provided by the server 110 may be installed in the first terminal device 120 and the second terminal device 130, and the remote meeting may be joined through the application. After the server 110 confirms that the first terminal device 120 has the editing permission, the server 110 confirms the activation of remote collaboration to the first interactive interface 122. After that, the first interactive interface 122 may send its operation information to the server 110, and the server 110 may then send the operation information to the second interactive interface 132. Explanation is further exemplified below.

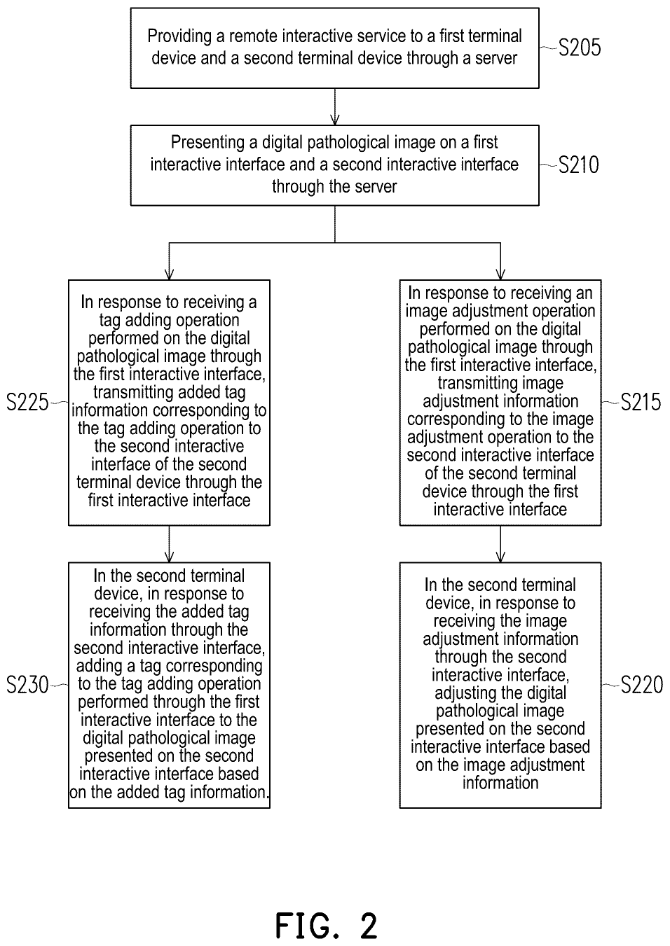

FIG. 2 is a flowchart illustrating a method for sharing a digital pathological image according to an embodiment of the disclosure. With reference to FIG. 1 and FIG. 2, in step S205, the server 110 provides the remote interactive service to the first terminal device 120 and the second terminal device 130. Specifically, the first terminal device 120 and the second terminal device 130 may connect the server 110 through wired or wireless communication protocols, thereby displaying the first interactive interface 122 and the second interactive interface 132 on displays of the first terminal device 120 and the second terminal device 130, respectively.

In step S210, the server 110 presents a digital pathological image on the first interactive interface 122 and the second interactive interface 132. Here, the digital pathological image is, for instance, uploaded to the server 110 by the editor through the first interactive interface 122 of the first terminal device 120, and then the server 110 presents the digital pathological image on the second interactive interface 132 of the second terminal device 130. In other embodiments, the digital pathological image may also be uploaded to the server 110 through the second terminal device 130 or other terminal devices, and after the editor makes selections from the server 110, the server 110 presents the digital pathological image on the first interactive interface 122 and the second interactive interface 132.

It is assumed that the digital pathological image is selected through the first terminal device 120, and after the digital pathological image is opened through the first interactive interface 122, an image format of the digital pathological image may be further converted into a standard format by the first interactive interface 122. The standard format includes, for instance, the DICOM format. That is, the interactive interface used by the terminal device providing the digital pathological image converts the image format of the digital pathological image into the standard format.

Next, in step S215, in response to receiving an image adjustment operation performed on the digital pathological image through the first interactive interface 122, image adjustment information corresponding to the image adjustment operation is transmitted to the second interactive interface 132 of the second terminal device 130 through the first interactive interface 122. For instance, the first interactive interface 122 first records the image adjustment information in a temporary storage region of the first terminal device 120 and then transmits the image adjustment information to the server 110, and finally, the server 110 transmits the image adjustment information to the second interactive interface 132.

Subsequently, in step S220, in the second terminal device 130, in response to receiving the image adjustment information through the second interactive interface 132, the digital pathological image presented on the second interactive interface 132 is correspondingly adjusted based on the image adjustment information.

In step S225, in response to receiving a tag adding operation performed on the digital pathological image through the first interactive interface 122, added tag information corresponding to the tag adding operation is transmitted to the second interactive interface 132 of the second terminal device 130 through the first interactive interface 122. After that, in step S230, in the second terminal device 130, in response to receiving the added tag information through the second interactive interface 132, a tag corresponding to the tag adding operation performed through the first interactive interface 122 is added to the digital pathological image presented on the second interactive interface 132 based on the added tag information. Further examples will be provided below for clarification.

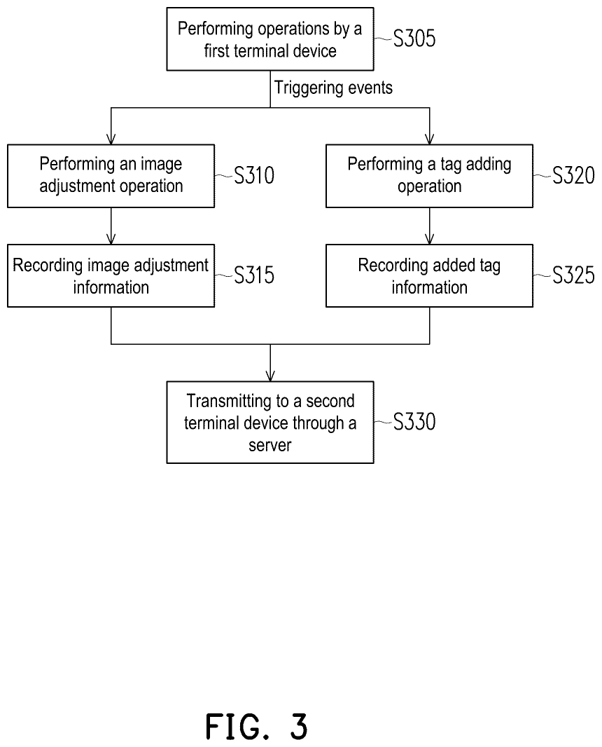

FIG. 3 is a flowchart illustrating a method of operating collaborative synchronization according to a first embodiment of the disclosure. With reference to FIG. 1 and FIG. 3, in step S305, the first terminal device 120 performs operations to trigger corresponding events. In this embodiment, the events triggered by the operations performed through the first interactive interface 122 include at least one of the following: step S310 and step S320.

In step S310, an image adjustment operation is performed on the digital pathological image through the first terminal device 120. Specifically, a user may perform the image adjustment operations including moving, zooming, and rotating the digital pathological image through the first interactive interface 122.

Next, in step S315, in response to the image adjustment operation (such as moving, zooming, rotating, etc.) performed on the digital pathological image through the first interactive interface 122, the image adjustment information is recorded through the first interactive interface 122 in the first terminal device 120. After that, in step S330, the image adjustment information is transmitted to the second terminal device 130 through the server 110. That is, the first interactive interface 122 of the first terminal device 120 transmits the image adjustment information to the second interactive interface 132 of the second terminal device 130 through the server 110. Here, the image adjustment information includes at least one of the following: a coordinate location of a middle point of the digital pathological image relative to a display frame of the first interactive interface 122, a zoom ratio for zooming the digital pathological image, and a rotation angle for rotating the digital pathological image.

For instance, a record name of the image adjustment information is set as “Operate_map”, and a record format of the image adjustment information may be “{coordinate location}; zoom ratio; rotation angle”. For instance: “{0471.1554, 33432.3224}; 2.0251; −1.5707”, where {0471.1554, 33432.3224} is the coordinate location, 2.0251 is an image zoom-in ratio, and −1.5707 is the rotation angle. The two values of the coordinate location represent x and y coordinates, respectively, while the zoom ratio and rotation angle are single numerical values.

On the other hand, in step S320, a tag adding operation is performed on the digital pathological image through the first terminal device 120. That is, the user may add tags to the digital pathological image through the first interactive interface 122.

Next, in step S325, in the first terminal device 120, in response to the tag adding operation performed on the digital pathological image through the first interactive interface 122, added tag information is recorded through the first interactive interface 122. After that, in step S330, the added tag information is transmitted to the second terminal device 130 through the server 110. That is, the first interactive interface 122 of the first terminal device 120 transmits the added tag information to the second interactive interface 132 of the second terminal device 130 through the server 110. Here, the added tag information includes at least one of the following: a tag identification code, a tag title, a tag descriptor, a tag length, a tag author, tag coordinate information, and a tag time.

The tag length represents a length of a line drawn on the digital pathological image. The tag length is a string, which may represent different units and have a value when a measurement function is annotated. The tag coordinate information is an array of polygons; that is, there are a plurality of x and y coordinates; otherwise, the tag descriptor is null. The tag title is filled in with the name of each nidus and automatically incremented (such as: _0, _1, _2 . . . ). For instance, if the nidus is breast cancer, it may be annotated as “Breast(DeleteArea)”, “Breast(A:Normal)”, “Breast(B:CIS)” or “Breast(C:IBC,NST)”. If the nidus is oral cancer, it may be annotated as “Oral(DeleteArea)”, “Oral(A:Normal)”, “Oral(B:Dysplasia)” or “Oral(C:SCC)”. If the nidus is thyroid cancer, it may be annotated as “Thyroid(DeleteArea)”, “Thyroid(A:PTC)”, “Thyroid(B:Non-tumor area)” or “Thyroid(C:Fibrosis/inflammatory stroma)”.

For instance, the record name of the added tag information is set as “Add_annotation”, and the record format of the added tag information may be “tag identification code; tag title; tag descriptor; tag length; tag author; tag coordinate information; tag time”. The tag identification code serves to represent the tag, e.g., “0_MeasureLayer_XXX”. The tag title may be “Thyroid(A:PTC)_0”. The tag descriptor serves to describe the nidus corresponding to the tag. The tag length may be “12.14 mm”. The tag author may be “Dr. Chen”. The tag coordinate information may be [[19356.7447, 31946.4414]; [25135.17082, 25672.7216]]. The tag time may be 1687140940566.

The operation information recorded by the first interactive interface 122 is transmitted to each terminal device set as the observer (e.g., the second terminal device 130), and each terminal device set as the observer then adjusts the content presented on its interactive interface (e.g., the second interactive interface 132) in synchronization according to the operation information.

For instance, the second terminal device 130, in response to the image adjustment information received through the second interactive interface 132, adjusts the digital pathological image presented on the second interactive interface 132 based on the image adjustment information. On the other hand, the second terminal device 130, in response to the added tag information received through the second interactive interface 132, adds a tag to the digital pathological image presented on the second interactive interface 132, which corresponds to the tag adding operation performed on the first interactive interface 122.

In an embodiment, the editor may manually tag different nidus locations, such as breast cancer, oral cancer, thyroid lesions, etc. In another embodiment, the interactive interface of the editor (e.g., the first interactive interface 122) is designed to quickly and continuously auto-select the nidus location. For instance, the server 110 provides an artificial intelligence (AI) module. In response to the input of the digital pathological image through the first terminal device 120, the digital pathological image is transmitted to the server 110 through the first interactive interface 122, so as to allow the AI module to automatically identify the nidus location in the digital pathological image. Moreover, in the first terminal device 120, in response to the tag adding operation performed on the digital pathological image through the first interactive interface 122, if the location of the tag adding operation falls within the nidus location selected by the AI module, the first interactive interface 122 automatically provides nidus information associated with the nidus location as the added tag information. As such, the editor may operate without interruption when selecting the same category as determined by the AI module, thus making the tagging operation more efficient. In an embodiment of the disclosure, if the location of the tag adding operation falls within the nidus location selected by the AI module, the nidus location selected by the AI module serves as one of the added tag information. The added tag information may be a category in the tag title, for instance, if the AI module determines the nidus location to be breast cancer, the category of the added tag title is automatically provided as “Breast”. If the AI module determines the nidus location to be oral cancer, the category of the added tag title is “Oral”. If the AI module determines the nidus location to be thyroid cancer, the category of the added tag title is “Thyroid”. In addition, the first interactive interface 122 may further use the tagged data as training data and send the data to the AI module for training, thus enabling the AI module to obtain identification results more accurately.

The server 110 provides a viewer (e.g., the first interactive interface 122 and the second interactive interface 132) for observing digital pathological images based on the standard DICOM format, and the viewer features a standardized AI tagging method and presents tiles and coordinates after AI tagging. It allows interactive and visualized presentation of digital pathological images and AI analysis results.

Since different manufacturers have different scanning formats for glass slides, according to the previous embodiment, the digital pathological images provided by various scanners may be uniformly converted into a unified DICOM format and presented on the viewer. The annotations made on the digital pathological images are stored and presented in the format of DICOM structured reporting (SR). The DICOM SR format is an international standard that allows the annotation results to be opened with different tools, thus eliminating the issue of being unable to open the results due to different data formats. Moreover, since the DICOM SR format is a universal format, it does not require additional document interpretation and still allows people to understand the meaning of each field, thus avoiding unclear data content.

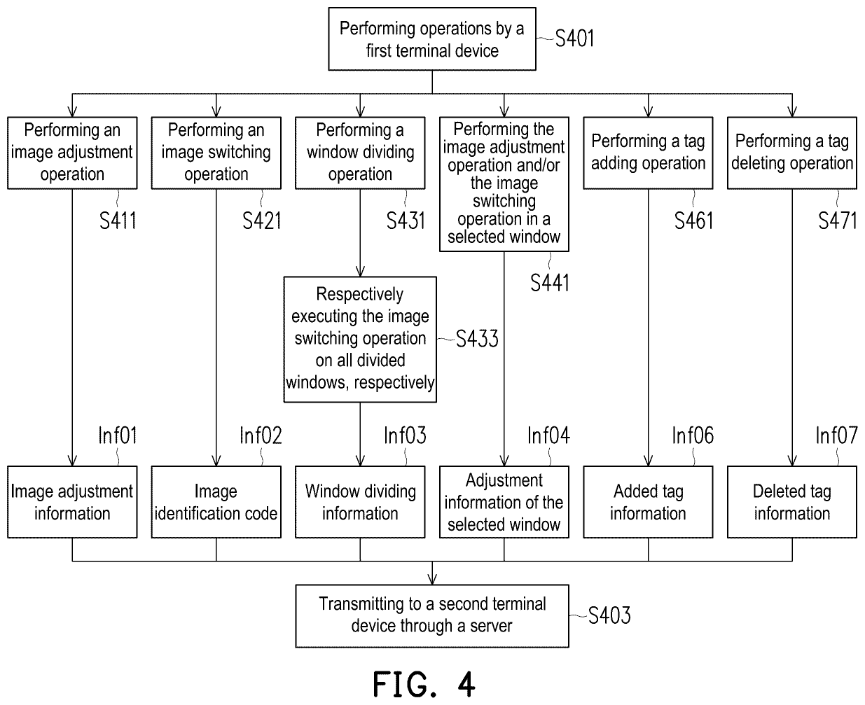

FIG. 4 is a flowchart illustrating a method of operating collaborative synchronization according to a second embodiment of the disclosure. With reference to FIG. 1 and FIG. 4, in step S401, the operation is performed by the first terminal device 120. The events triggered by the operations through the first interactive interface 122 include at least one of the following: step S411, step S421, step S431, step S441, step S451, step S461, and step S471.

In step S411, an image adjustment operation is performed on the digital pathological image through the first interactive interface 122 of the first terminal device 120, and image adjustment information Inf01 is recorded through the first interactive interface 122. Detailed explanations of step S411 may be referred to as the explanations of steps S310 and S315 and will not be elaborated hereinafter.

In step S421, an image switching operation is performed on the digital pathological image presented on the first interactive interface 122. For instance, another digital pathological image is selected through the first interactive interface 122, and an image identification code Inf02 of the selected digital pathological image is recorded. After that, in step S403, the recorded information is transmitted to the second terminal device 130 through the server 110. In terms of the operation in step S421, in step S403, the first interactive interface 122 transmits the image identification code Inf02 to the second interactive interface 132 of the second terminal device 130 through the server 110. Accordingly, the second interactive interface 132 synchronously switches the digital pathological image displayed on the second interactive interface 132 based on the image identification code Inf02.

In step S431, a window dividing operation is performed through the first interactive interface 122, and in step S433, an image switching operation is executed on all divided windows through the first interactive interface 122. After that, based on the window dividing operation and the image switching operation, window dividing information Inf03 is recorded through the first interactive interface 122. The window dividing information Inf03 includes the number of the divided windows, a window identification code of each of the divided windows, and image identification codes of the digital pathological images presented on the divided windows.

In an embodiment, a display frame of the first interactive interface 122 may be divided into two divided windows, four divided windows, or the like. Each of the divided window may display one digital pathological image. The digital pathological images displayed by the divided windows may be the same image (displaying the same or different parts) or different images.

For instance, a record format of the window dividing information Inf03 may be “the number of the divided windows; [window identification code 1, window identification code 2, . . . ]; [image identification code 1, image identification code 2, . . . ]”. If it is assumed that the number of the divided windows is four, the window dividing information Inf03 is, for instance, “4; [W001, W002, W003, W004]; [Img001, Img002, Img003, Img004]”. Here, the window identification codes and the image identification codes are recorded in a manner of an array; for instance, according to the locations of the divided windows, the corresponding window identification codes and image identification codes are recorded in the order of top left, top right, bottom left, and bottom right locations.

In the second terminal device 130, in response to receiving the window dividing information Inf03 through the second interactive interface 132, a plurality of divided windows are obtained by performing a window dividing operation on the second interactive interface 132 based on the window dividing information Inf03, and the corresponding digital pathological image is presented in each of the divided windows.

After a window dividing function is enabled, i.e., after the window dividing operation is performed through the first interactive interface 122 and the divided windows are obtained, the first interactive interface 122 may further enable a lock function. In the first terminal device 120, after the lock function is enabled through the first interactive interface 122, in response to performing the image adjustment operation on one of the divided windows through the first interactive interface 122, the digital pathological images presented in at least one of the other divided windows are correspondingly adjusted, and the image adjustment information is recorded and transmitted to the second interactive interface 132 of the second terminal device 130 through the first interactive interface 122, so as to correspondingly adjust the digital pathological images presented on the second interactive interface 132 based on the image adjustment information.

In other words, when the lock function is enabled, the divided windows may perform synchronized rotation, movement, zooming, and other image adjustment operations, which facilitates the user to compare the digital pathological images. Since the lock function allows the operations of the divided windows to be synchronously locked, the operations under the lock function simultaneously trigger (record) a plurality of image adjustment information.

In step S441, the image adjustment operation and/or the image switching operation in the selected window is performed. Specifically, one of the divided windows in the first interactive interface 122 is selected as the selected window. In response to the image adjustment operation and/or the image switching operation performed on the digital pathological image presented in the selected window through the first interactive interface 122, adjustment information Inf04 of the selected window is recorded through the first interactive interface 122, and the adjustment information Inf04 of the selected window (the image adjustment information and/or the image identification code of the selected window) is transmitted to the second interactive interface 132 of the second terminal device 130 through the first interactive interface 122.

After the window dividing operation is performed on the second terminal device 130 to obtain the divided windows, in response to receiving the adjustment information Inf04 of the selected window through the second interactive interface 132, the digital pathological image presented in the selected window corresponding to the second interactive interface is adjusted based on the adjustment information Inf04 of the selected window through the second interactive interface 132.

In step S461, a tag adding operation is performed on the digital pathological image through the first terminal device 120, and added tag information Inf06 is recorded. Detailed explanations of step S461 may be referred to as the explanations of steps S320 and S325 and will not be elaborated hereinafter.

In step S471, a tag deleting operation is performed on the digital pathological image through the first terminal device 120, and deleted tag information Inf07 is recorded. After that, in step S403, the first interactive interface 122 sends the deleted tag information Inf07 to the second interactive interface 132 of the second terminal device 130 through the server 110. Here, the deleted tag information includes a tag identification code corresponding to the deleted tag. In the second terminal device 130, in response to receiving the deleted tag information through the second interactive interface 132, the corresponding tag is removed from the digital pathological image presented on the second interactive interface 132 based on the deleted tag information through the second interactive interface 132.

Besides, after the divided windows, are obtained, one of the divided windows may be selected as the selected window, and the operation in step S461 may be performed on the digital pathological image presented in the selected window. In response to the tag adding operation performed on the digital pathological image presented in the selected window through the first interactive interface 122, the added tag information is recorded through the first interactive interface 122, and the added tag information is transmitted to the second interactive interface 132 of the second terminal device 130 through the first interactive interface 122. In the second terminal device 130, in response to receiving the added tag information through the second interactive interface 132, a tag corresponding to the tag adding operation performed through the first interactive interface 122 is added to the digital pathological image presented in the selected window corresponding to the second interactive interface 132 based on the added tag information.

Alternatively, after the divided windows are obtained, one of the divided windows may be selected as the selected window, and the operation in step S471 may be performed on the digital pathological image presented in the selected window. Accordingly, when an editor deletes one of the tags in the selected window through the first interactive interface 122, the corresponding tag is removed simultaneously from the selected window corresponding to the second interactive interface 132.

In addition to the tagging function, the system provided in one or more embodiments of the disclosure allows multi-slide observations and editing of digital pathological images that are on the same glass slide but have different stains through the window dividing operation. Combined with the lock function, the rotation, translational movement, and zooming of some or all of the glass slides in the divided windows on the screen are synchronized, which facilitates the user to compare two or more glass slides simultaneously on the conditions of the same angle, displacement, and zoom level. Besides, the interactive interface provided in one or more embodiments of the disclosure may be used on different terminal devices; for instance, the observing function may be performed on the terminal devices including desktop computers, laptop computers, tablet computers, and even smartphones.

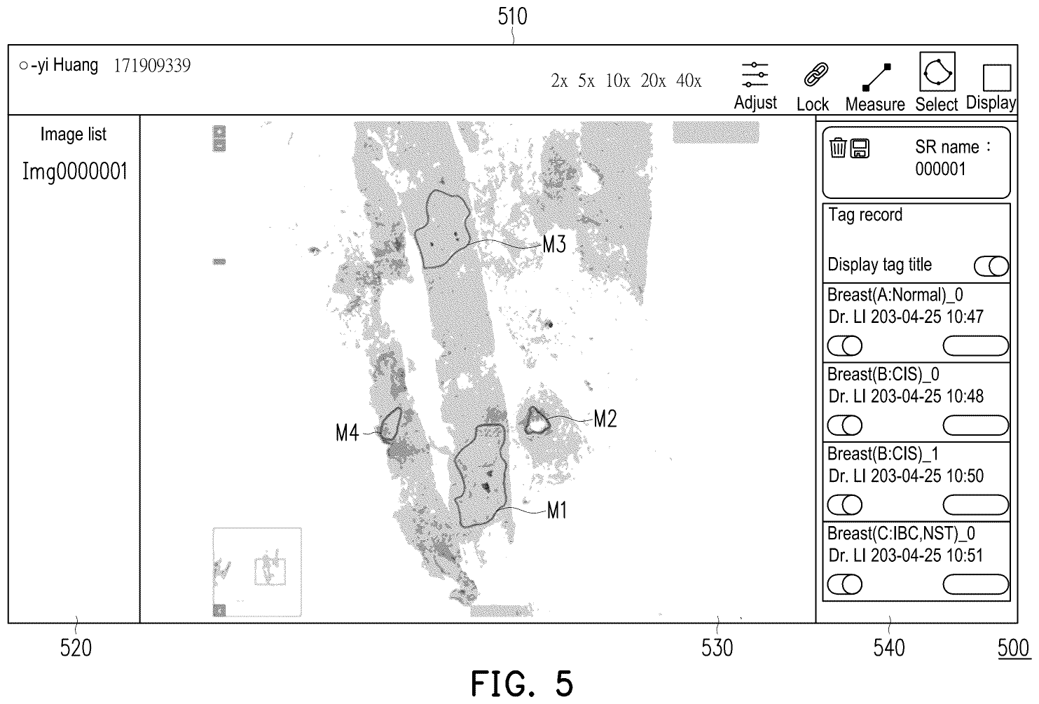

FIG. 5 is a schematic view illustrating an interactive interface according to an embodiment of the disclosure. With reference to FIG. 5, an interactive interface 500 includes four sections (sections 510, 520, 530, and 540). The section 510 serves to display patient information corresponding to the digital pathological image and provide a plurality of editing functions. The editing functions in the section 510 are exclusively provided to the terminal devices with the editing permissions. The section 520 serves to provide an image list for the editor to select the digital pathological image, and the selected digital pathological image is presented in the display frame in the section 530. The section 540 serves to present an SR name and a tag record corresponding to an image Img0000001. Here, the SR name refers to a tag name to be recorded by the editor at present. For instance, after the editor selects the region to be tagged, “000001” is entered as the tag name.

The editing functions include an image zooming function, an adjustment function, a lock function, a measurement function, a selection function, a display function, and so on. For instance, zoom ratios of the image zooming function include 2×, 5×, 10×, 20×, 40×, and so on. The adjustment function is performed for moving the digital pathological image presented in the section 530, rotating the digital pathological image presented in the section 530, or carrying out other operations on the digital pathological image presented in the section 530. The measurement function and the selection function serve to manually tag the digital pathological image presented in the section 530. For instance, tags M1-M4 are added to the digital pathological image through performing the selection function. The display function serves to determine whether to display the information of all of the tags in the digital pathological image in the section 530.

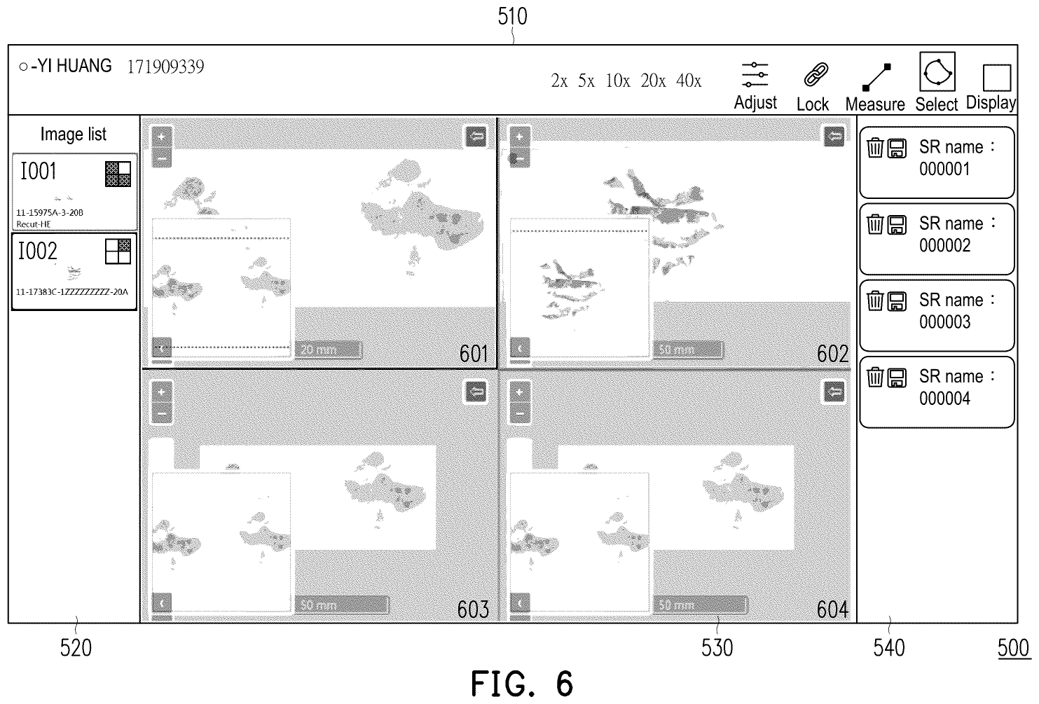

FIG. 6 is a schematic view illustrating divided windows according to an embodiment of the disclosure. With reference to FIG. 6, in this embodiment, the section 530 of the interactive interface 500 is divided into four, i.e., divided windows 601, 602, 603, and 604. The section 520 displays thumbnails I001 and I002 of two digital pathological images. The section 540 displays the SR names corresponding to divided windows 601, 602, 603, and 604, respectively. Here, the SR names “000001”-“000004” represent tag records corresponding to the divided windows 601, 602, 603, and 604, respectively.

After the divided windows 601, 602, 603, and 604 are obtained, the editor may drag the required digital pathological image from the section 520 to the corresponding divided window in the section 530. In the thumbnails I001 and I002 in the section 520, a grid (which may be double or quadruple grids, depending on the current divided window) is applied to indicate the divided window to which the digital pathological image is dragged. In this embodiment, 2×2 grids are applied, where the parts filled with black dots indicate the location of the divided window to which the digital pathological image has been dragged. That is, the digital pathological image corresponding to the thumbnail I001 is selected for the divided windows 601, 603, and 604, while the digital pathological image corresponding to the thumbnail I002 is selected for the divided window 602.

After the divided windows 601-604 are obtained, the lock function may be performed to synchronously rotate, move, and zoom the digital pathological images presented on at least two divided windows, and other image adjustment operations may also be performed on the digital pathological images presented on at least two divided windows.

Besides, the interactive interface further provides a poll function to obtain consensus from a plurality of participants, which may further improve the accuracy of the tagging operation.

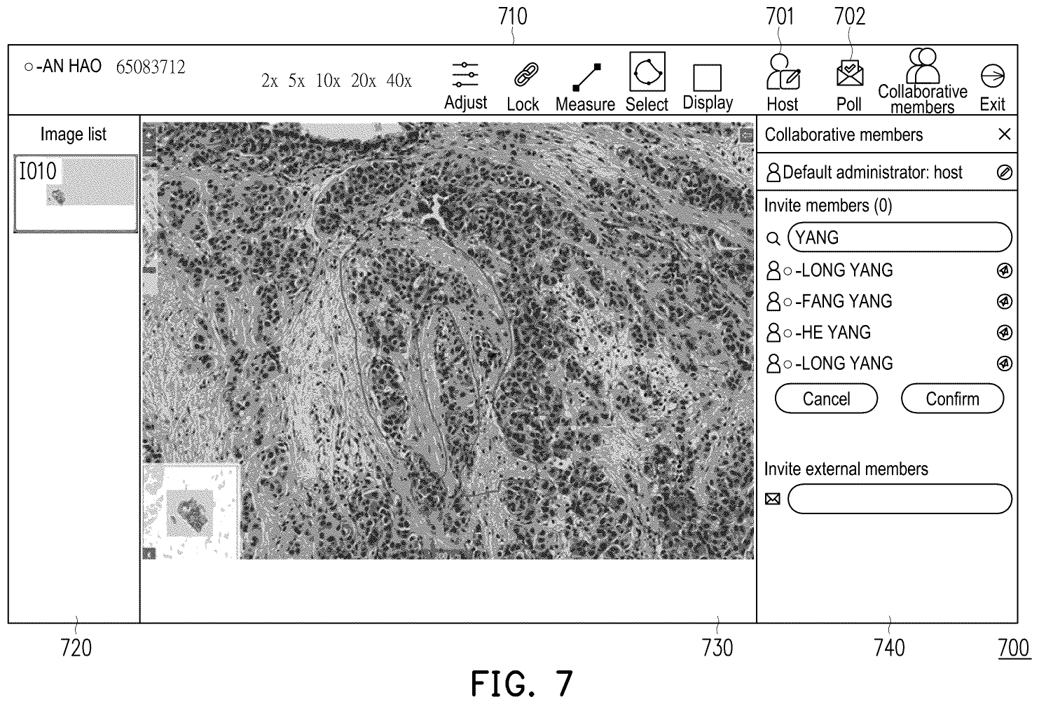

FIG. 7 is a schematic view illustrating an interactive interface having an editing permission according to an embodiment of the disclosure. As shown in FIG. 7, an image 701 indicates that the interactive interface 700 has the editing permission at this time. The host (the meeting creator) may perform a member inviting function of the interactive interface 700 to find participants to join the meeting, and after a “confirm” button is clicked, the host may wait for participants to join the meeting. In addition, after other participants join the meeting, the host (the meeting creator) may further decide which participant to whom the editing permission is to be switched.

An interactive interface 700 includes sections 710, 720, 730, and 740. The section 710 serves to display the patient information and the image 701 corresponding to the digital pathological image and provide a plurality of editing functions and a poll function 702. The section 720 serves to provide an image list for the editor to select the digital pathological image, and the selected digital pathological image is presented in the section 730. The section 740 serves to select a list of collaborative members (observers).

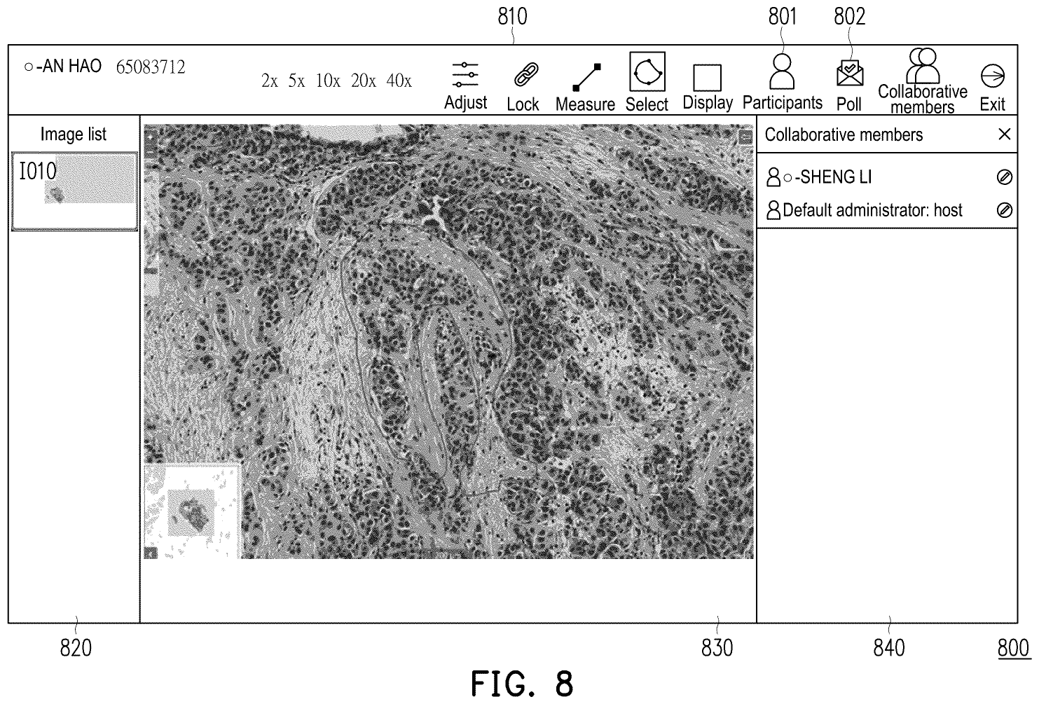

FIG. 8 is a schematic view illustrating an interactive interface having no editing permission according to an embodiment of the disclosure. In FIG. 8, an interactive interface 800 may be viewed, but the content presented on the interactive interface 800 cannot be edited, nor can the editing permission be changed. The interactive interface 800 includes sections 810, 820, 830, and 840. The section 810 serves to display patient information and image 801 corresponding to the digital pathological image and provide a plurality of editing functions and a poll function 802. The section 820 serves to provide an image list for the editor to select the digital pathological image, and the selected digital pathological image is presented in the section 830. The section 840 serves to display a list of collaborative members (editors or observers).

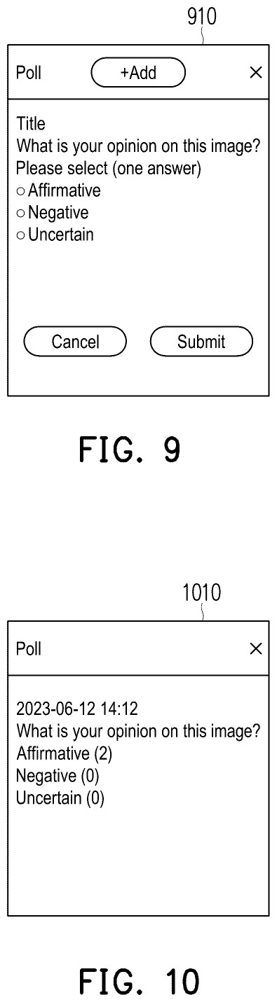

When the number of the participants in the meeting reaches two or more, the poll function 702 on the interactive interface 700 may be enabled. In response to enabling the poll function 702, a poll menu page is created and displayed in the section 740 for the editor to create a poll menu. Here, the poll menu includes a plurality of options. FIG. 9 is a schematic view illustrating a poll menu according to an embodiment of the disclosure. With reference to FIG. 9, a poll title of the poll menu 910 is “What is your opinion on this image?”, which includes three options (affirmative, negative, uncertain).

After the poll menu 910 is created, the interactive interface 700 sends the poll menu 910 to other interactive interfaces 800 with no editing permission through the server 110. Thereby, the participants may select one of the options on the terminal device they are using through the interactive interface 800 and send the poll information to the server. The poll information includes a poll number (e.g., the number of the meeting creator or the participant), a poll time, the poll title, and a poll answer (e.g., affirmative, negative, or uncertain). Moreover, the server 110 receives the poll information from the interactive interface 800 and presents the obtained poll results to each terminal device.

FIG. 10 is a schematic view illustrating a poll result according to an embodiment of the disclosure. A poll result 1010 indicates that the meeting creator and the participant have the same consensus.

To sum up, the method and the system for sharing the digital pathological images as provided in one or more embodiments of the disclosure may be used in the application of remote consultation and collaborative synchronization and may support standard file format pathological images, allow multi-party synchronous interactions, and ensure smooth image transmissions. According to the method and the system for sharing the digital pathological images as provided in one or more embodiments of the disclosure, different pathological conditions, such as breast cancer, oral cancer, thyroid lesions, and so on, may be classified and tagged through the interactive interface. The interactive interface may also be designed to quickly and continuously auto-select the nidus location in the digital pathological image; accordingly, when the editor selects the same category, the operation is not interrupted, thus making the tagging operation more efficient.

No matter whether the tag information refers to manual tagging, AI training, or AI inference results, the DICOM SR format is applied herein to store the tag information, thereby preventing the generation of incomprehensible data content and reducing the occurrence of errors. The universality of the general format is no longer limited to a single tool. The DICOM SR provided in the method and the system for sharing the digital pathological images according to one or more embodiments of the disclosure is also subject to strict confidentiality obligations, such as setting permission management, anonymization, and so on, so as to prevent the DICOM SR from being exposed to general users and thereby maintain data security.

In the method and the system for sharing the digital pathological images according to one or more embodiments of the disclosure, the poll function is further provided to obtain consensus among doctors from various departments, thereby obtaining accurate diagnosis results and treatment methods.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A method for sharing a digital pathological image, the method comprising:
providing a remote interactive service to a first terminal device and a second terminal device through a server, wherein the first terminal device comprises a first interactive interface, the second terminal device comprises a second interactive interface, the first terminal device has an editing permission, and the second terminal device has an observing permission;
presenting the digital pathological image to the first interactive interface and the second interactive interface through the server;
in response to receiving an image adjustment operation performed on the digital pathological image through the first interactive interface, transmitting image adjustment information corresponding to the image adjustment operation to the second interactive interface of the second terminal device through the first interactive interface;
in response to receiving a tag adding operation performed on the digital pathological image through the first interactive interface, transmitting added tag information corresponding to the tag adding operation to the second interactive interface of the second terminal device through the first interactive interface;
in the second terminal device, in response to receiving the image adjustment information through the second interactive interface, adjusting the digital pathological image presented on the second interactive interface based on the image adjustment information; and
in the second terminal device, in response to receiving the added tag information through the second interactive interface, adding a tag corresponding to the tag adding operation performed through the first interactive interface to the digital pathological image presented on the second interactive interface based on the added tag information.

2. The method as claimed in claim 1, further comprising:
performing a window dividing operation through the first interactive interface and obtaining a plurality of divided windows;
performing an image switching operation on each of the divided windows through the first interactive interface and presenting the corresponding digital pathological images in each of the divided windows;
based on the window dividing operation and the image switching operation, recording window dividing information through the first interactive interface, wherein the window dividing information comprises the number of the divided windows, a window identification code of each of the divided windows, and image identification codes of the digital pathological images presented in the divided windows;
transmitting the window dividing information to the second interactive interface of the second terminal device through the first interactive interface; and
in the second terminal device, in response to receiving the window dividing information through the second interactive interface, performing the window dividing operation through the second interactive interface based on the window dividing information to obtain the divided windows, and presenting the corresponding digital pathological images in each of the divided windows.

3. The method as claimed in claim 2, wherein after performing the window dividing operation through the first interactive interface and obtaining the divided windows, the method further comprising:
in a selected window of the divided windows on the first interactive interface, in response to receiving the image adjustment operation performed on the digital pathological image presented in the selected window through the first interactive interface, transmitting the image adjustment information corresponding to the image adjustment operation performed on the selected window to the second interactive interface of the second terminal device through the first interactive interface; and
in response to receiving the image adjustment information through the second interactive interface, based on the image adjustment information, adjusting the digital pathological image presented in the selected window corresponding to the second interactive interface through the second interactive interface.

4. The method as claimed in claim 2, wherein after performing the window dividing operation through the first interactive interface and obtaining the divided windows, the method further comprising:
in a selected window of the divided windows on the first interactive interface, in response to the tag adding operation performed on the digital pathological image presented in the selected window through the first interactive interface, recording the added tag information through the first interactive interface, and transmitting the added tag information to the second interactive interface of the second terminal device through the first interactive interface;
in response to receiving the added tag information through the second interactive interface, adding the tag corresponding to the tag adding operation performed through the first interactive interface to the digital pathological image presented in the selected window corresponding to the second interactive interface through the second interactive interface based on the added tag information.

5. The method as claimed in claim 2, wherein after performing the window dividing operation through the first interactive interface and obtaining the divided windows, the method further comprising:
in the first terminal device, after enabling a lock function through the first interactive interface, in response to the image adjustment operation performed on at least one of the divided windows through the first interactive interface, synchronously adjusting the digital pathological images presented in the other divided windows of the divided windows, and recording and transmitting the image adjustment information to the second interactive interface of the second terminal device through the first interactive interface, so as to synchronously adjust the corresponding digital pathological images presented on the second interactive interface through the second interactive interface based on the image adjustment information.

6. The method as claimed in claim 1, wherein after transmitting the added tag information corresponding to the tag adding operation to the second interactive interface of the second terminal device through the first interactive interface, the method further comprising:
in response to a tag deleting operation performed on the digital pathological image through the first interactive interface, transmitting deleted tag information corresponding to the tag deleting operation to the second interactive interface of the second terminal device through the first interactive interface, wherein the deleted tag information comprises at least one of a tag identification code, a tag title, a tag descriptor, a tag length, a tag author, tag coordinate information, and a tag time, wherein
in the second terminal device, in response to receiving the deleted tag information through the second interactive interface, removing the corresponding tag from the digital pathological image presented on the second interactive interface through the second interactive interface based on the deleted tag information.

7. The method as claimed in claim 1, wherein the digital pathological image is provided by the first terminal device or the second terminal device, and an image format of the digital pathological image is converted into a standard format through the first interactive interface or the second interactive interface adopted by the first terminal device or the second terminal device providing the digital pathological image.

8. The method as claimed in claim 1, further comprising:
providing an artificial intelligence module through the server;
receiving the digital pathological image through the artificial intelligence module to identify a nidus location in the digital pathological image by the artificial intelligence module;
in response to the tag adding operation performed on the digital pathological image through the first interactive interface and in response to a location where the tag adding operation is performed falling within the nidus location, providing nidus information associated with the nidus location as the added tag information through the first interactive interface.

9. The method as claimed in claim 1, further comprising:
in response to the first terminal device having the editing permission, enabling a poll function, establishing a poll menu and sending the poll menu to the server by the first terminal device, wherein the poll menu comprises a plurality of options;
after displaying the poll menu in the first terminal device and the second terminal device through the server, sending poll information to the server by the first terminal device and the second terminal device according to the poll menu, wherein the poll information comprises a poll number, a poll time, a poll title, and a poll answer;
receiving the poll information through the server, counting the poll information to obtain a poll result, and displaying the poll result through the first terminal device or the second terminal device.

10. The method as claimed in claim 1, wherein the image adjustment information comprises at least one of a coordinate location of a middle point of the digital pathological image relative to a display frame of the first interactive interface, a zoom ratio for zooming the digital pathological image, and a rotation angle for rotating the digital pathological image,
wherein the added tag information comprises at least one of a tag identification code, a tag title, a tag descriptor, a tag length, a tag author, tag coordinate information, and a tag time.

11. A system for sharing a digital pathological image, comprising:
a server, providing a remote interactive service;
a first terminal device, having an editing permission and comprising a first interactive interface provided by the remote interactive service; and
a second terminal device, having an observing permission and comprising a second interactive interface provided by the remote interactive service,
wherein the server presents the digital pathological image to the first interactive interface and the second interactive interface,
the first terminal device is configured to:
in response to receiving an image adjustment operation performed on the digital pathological image through the first interactive interface, transmit image adjustment information corresponding to the image adjustment operation to the second interactive interface of the second terminal device through the first interactive interface;
in response to receiving a tag adding operation performed on the digital pathological image through the first interactive interface, transmit added tag information corresponding to the tag adding operation to the second interactive interface of the second terminal device through the first interactive interface,
and the second terminal device is configured to:
in response to receiving the image adjustment information through the second interactive interface, adjust the digital pathological image presented on the second interactive interface based on the image adjustment information; and
in response to receiving the added tag information through the second interactive interface, add a tag corresponding to the tag adding operation performed through the first interactive interface to the digital pathological image presented on the second interactive interface based on the added tag information.

12. The system as claimed in claim 11, wherein the first terminal device is configured to:
perform a window dividing operation through the first interactive interface and obtaining a plurality of divided windows;
perform an image switching operation on each of the divided windows through the first interactive interface and presenting the corresponding digital pathological images in each of the divided windows;
based on the window dividing operation and the image switching operation, record window dividing information through the first interactive interface, wherein the window dividing information comprises the number of the divided windows, a window identification code of each of the divided windows, and image identification codes of the digital pathological images presented in the divided windows; and
transmit the window dividing information to the second interactive interface of the second terminal device through the first interactive interface,
wherein the second terminal device is configured to:
in response to receiving the window dividing information through the second interactive interface, perform the window dividing operation through the second interactive interface based on the window dividing information to obtain the divided windows, and present the corresponding digital pathological images in each of the divided windows.

13. The system as claimed in claim 12, wherein
after the first terminal device performs the window dividing operation through the first interactive interface and obtains the divided windows, in a selected window of the divided windows on the first interactive interface, in response to receiving the image adjustment operation performed on the digital pathological image presented in the selected window through the first interactive interface, the first terminal device transmits the image adjustment information corresponding to the image adjustment operation on the selected window to the second interactive interface of the second terminal device through the first interactive interface; and
after performing the window dividing operation and obtaining the divided windows, in response to receiving the image adjustment information through the second interactive interface, based on the image adjustment information, the second terminal device adjusts the digital pathological image presented in the selected window corresponding to the second interactive interface through the second interactive interface.

14. The system as claimed in claim 12, wherein
after the first terminal device performs the window dividing operation through the first interactive interface and obtains the divided windows, in a selected window of the divided windows on the first interactive interface, in response to the tag adding operation performed on the digital pathological image presented in the selected window through the first interactive interface, the first terminal device records the added tag information through the first interactive interface, and transmits the added tag information to the second interactive interface of the second terminal device through the first interactive interface;
after performing the window dividing operation and obtaining the divided windows, in response to receiving the added tag information through the second interactive interface, the second terminal device adds the tag corresponding to the tag adding operation performed through the first interactive interface to the digital pathological image presented in the selected window corresponding to the second interactive interface through the second interactive interface based on the added tag information.

15. The system as claimed in claim 12, wherein
after the first terminal device performs the window dividing operation through the first interactive interface, obtains the divided windows, and enables a lock function, in response to the image adjustment operation performed on at least one of the divided windows through the first interactive interface, the first terminal device synchronously adjusts the digital pathological images presented in the other divided windows of the divided windows, and records and transmits the image adjustment information to the second interactive interface of the second terminal device through the first interactive interface, so as to synchronously adjust the corresponding digital pathological images presented on the second interactive interface through the second interactive interface based on the image adjustment information.

16. The system as claimed in claim 11, wherein after the first terminal device performs the tag adding operation through the first interactive interface, in response to a tag deleting operation performed on the digital pathological image through the first interactive interface, the first terminal device transmits deleted tag information corresponding to the tag deleting operation to the second interactive interface of the second terminal device through the first interactive interface, wherein the deleted tag information comprises at least one of a tag identification code, a tag title, a tag descriptor, a tag length, a tag author, tag coordinate information, and a tag time;
wherein in response to receiving the deleted tag information through the second interactive interface, the second terminal device removes the corresponding tag from the digital pathological image presented on the second interactive interface through the second interactive interface based on the deleted tag information.

17. The system as claimed in claim 11, wherein the digital pathological image is provided by the first terminal device or the second terminal device, and an image format of the digital pathological image is converted into a standard format through the first interactive interface or the second interactive interface adopted by the first terminal device or the second terminal device providing the digital pathological image.

18. The system as claimed in claim 11, wherein the server provides an artificial intelligence module, and the digital pathological image is received through the artificial intelligence module to identify a nidus location in the digital pathological image by the artificial intelligence module;
wherein the first terminal device is configured to, in response to the tag adding operation performed on the digital pathological image through the first interactive interface and in response to a location where the tag adding operation is performed falling within the nidus location, spontaneously provide nidus information associated with the nidus location as the added tag information through the first interactive interface.

19. The system as claimed in claim 11, wherein the first terminal device having the editing permission is configured to:
in response to enabling a poll function, establish a poll menu and sending the poll menu to the server, wherein the poll menu comprises a plurality of options,
after the server displays the poll menu in the first terminal device and the second terminal device, the first terminal device and the second terminal device are enabled to send poll information to the server according to the poll menu, wherein the poll information comprises a poll number, a poll time, a poll title, and a poll answer, and
the server receives the poll information, counts the poll information to obtain a poll result, and displays the poll result through the first terminal device or the second terminal device.

20. The system as claimed in claim 11, wherein the image adjustment information comprises at least one of a coordinate location of a middle point of the digital pathological image relative to a display frame of the first interactive interface, a zoom ratio for zooming the digital pathological image, and a rotation angle for rotating the digital pathological image,
wherein the added tag information comprises at least one of a tag identification code, a tag title, a tag descriptor, a tag length, a tag author, tag coordinate information, and a tag time.
